(19) United States
(12) Patent Application Publication
(10) Pub. No.: US 2022/0339403 A1
(43) Pub. Date: Oct. 27, 2022

(54) SEMIRIGID DRIVE SHAFT FOR ENDOSCOPIC PROBE

(71) Applicant: CANON U.S.A., INC., MELVILLE, NJ (US)

(72) Inventor: Xuri Yan, Newton, MA (US)

(21) Appl. No.: 17/763,484

(22) PCT Filed: Sep. 30, 2020
(86) PCT No.: PCT/US20/53597

§ 371 Date: Mar. 24, 2022

Related U.S. Application Data
(60) Provisional application No. 62/923,077, filed on Oct. 18, 2019.

(51) Int. Cl. A61M 25/00; A61M 25/01; A61B 1/005

(52) U.S. Cl. CPC A61M 25/0052; A61M 25/0108; A61B 1/0055

(57) ABSTRACT

A flexible tubular drive shaft for a medical device comprising: an elongated tubular body having an opening extending along a longitudinal axis from a proximal end and a distal end. The elongated tubular body includes a proximal portion having a first rigid section and a first flexible section; a middle portion having a second rigid section; and a distal portion having a second flexible section. The first rigid section is configured to couple the tubular drive shaft to a torque input apparatus to transfer torque to the distal end of the tubular body. The first flexible section is configured to minimize torsion of the tubular body between the first rigid section and the second rigid section, and the second flexible section is configured to increase flexibility of the distal portion of the tubular body.

### CROSS-REFERENCE TO RELATED APPLICATIONS

This application claims priority to U.S. Provisional Application No. 62/923,077, filed Oct. 18, 2019, the content of which is incorporated by reference herein in its entirety.

### BACKGROUND INFORMATION

### Field of Disclosure

The present disclosure generally relates to medical devices. More particularly, the disclosure relates to minimally invasive medical devices including a tubular drive shaft for rotating catheter or endoscopic probes applicable to surgical or imaging techniques, such as intravascular ultrasound (IVUS), optical coherence tomography (OCT), spectrally encoded endoscopy (SEE), and the like.

### Description of Related Art

Surgical and imaging techniques that use rotatable (and sometimes steerable) probes are dependent on the use of a flexible rotary shaft to transmit torque (rotating force) from a torque input device (motor) located at the proximal end to an imaging device or tool located at the distal end. These flexible rotary shafts include a single layer or multiple-layers of torque coils and slot cut metal tubes for the entire driving path which results in a long flexible rotary shaft. In addition, these rotary shafts are required to be very thin, so that they can be delivered through delicate anatomical paths, such as the vasculature, genitourinary tracts, respiratory tracts, and other such bodily lumens. Most surgical and imaging devices that use these flexible rotary shafts suffer certain level of irregularities caused by torsional strain as the flexible shaft is guided through tortuous anatomical paths. For example, torsional strain causes the rotation speed of the rotatable shaft at the distal end of the shaft to be different from the rotation speed input at the proximal end thereof. This occurs especially when the flexible shaft is delivered through tortuous anatomical paths, and at least in part of the rotating components experience friction against stationary components or the patient's anatomy, which leads to non-uniform rotational distortion (NURD). NURD is an imaging artifact known to cause significant image distortion.

To address the issues caused by NURD, various techniques have been disclosed in patent and non-patent literature. By way of example, pre-grant patent application publication US 2010/0249601 to Courtney cites various publications specifically directed to reducing NURD. Courtney particularly discloses an imaging probe having a frictional element integrated therewith for reducing non-uniform rotational distortion near the distal end. According to Courtney, one or more frictional components and a bearing are mounted in frictional contact with an inner surface of an elongate hollow sheath so that during rotation the rotational drive mechanism operates with a higher torsional load than in an absence of the one or more frictional components thereby reducing non-uniform rotational distortion. Adding the one or more frictional components and a bearing to the drive shaft may reduce NURD, but it would increase the manufacturing costs and would make the operation of the device more complicated.

U.S. Pat. No. 6,447,518 B1 to Krause et al., (Krause) discloses a flexible tubular shaft having a helical slot cut around and along the tubular wall. This flexible shaft offers different degrees of flexibility along the length of the shaft by having the pitch of the helical slot vary along the length of the shaft. The varied flexibility corresponds to the variation in the pitch of the helical slot. The helical path can have a helix angle in the range of about 10 to 45 degrees, and the helix angle can be varied along the length of the shaft to produce correspondingly varied flexibility. However, the diameter of this conventional drive shaft is in a range from about 0.15 to 4.00 inches; and the ratio of the inner diameter (ID) to the outer diameter (OD) of the shaft is in the range from about 1:1.2 to 1:4. At these dimensions, the flexible shaft disclosed by Krause is not practical for most minimally invasive surgical (MIS) procedures such as intravascular imaging and neurosurgical interventions.

Pre-grant patent application publication US 2019/0060612 by Besselink discloses a tubular sheath with one or more helical slots which purportedly improves flexibility and structural rigidity through variations in width and pitch of the slots along the length of the tubular sheath.

As another example, U.S. Pat. No. 8,932,235 to Jacobsen et al., (Jacobsen) discloses a drive shaft such as a guidewire applicable to intravascular imaging. According to Jacobsen, the guidewire includes a tubular member and a core wire arranged inside the tubular member such that the tubular member and the core wire share a common longitudinal axis. The tubular member has a first group of slots formed at the proximal end and a second group of slots formed at the distal end. The spacing between slots may be varied to change bending stiffness of the guidewire.

In the current state-of-the-art, techniques for making a flexible rotary tubular shaft to transmit torque from a proximal drive power unit (a motor) to an imaging device at the distal end of the probe mainly include two approaches. One is the use of single layer or multiple layered torque coils (as disclosed by Krause), and the other is the use of slot cut metal tubes (as disclosed by Besselink). If a torque coil is used as a drive shaft for an imaging probe, it constantly causes rotational NURD error which increases with the increase of the torque coil length and distorts the image. On the other hand, when using the slot cut metal tube as flexible drive shaft, in addition to the NURD issue, if the drive shaft needs to go through a very tight bending paths (small bending radius), the drive shaft will get fatigued quickly under the periodic bending motion when it is rotating. Thus the product life will be significantly reduced.

One or more embodiments of the present disclosure overcome deficiencies associated with conventional flexible rotary tubular shafts. Flexible rotary tubular shafts disclosed herein have sufficient torqueability, tensile strength, and flexibility to be used in a variety of applications for treating bodily lumens, including but not limited to intravascular lumens.

### SUMMARY OF EXEMPLARY EMBODIMENTS

To overcome disadvantages of the state-of-the-art and/or improve on conventional technology, the present disclosure proposes a novel integral, semi-rigid, and torsionally inflexible tubular drive shaft configured to accommodate within its inner diameter imaging components, such as optical fibers and micro-optical imaging components. The proposed semi-rigid hybrid drive shaft takes advantage of the flexibility of torque coil designs and low NURD rigid tube design to reduce imaging NURD errors by shortening the length of the flexible portion which is associated with NURD. The present disclosure provides a novel flexible tubular drive shaft to overcome the disadvantages of the state-of-the-art in imaging technologies using flexible rotary shaft if it goes through a relatively long straight driving path in addition to tortuous and tightly bend paths.

According to various embodiments, a flexible tubular drive shaft (100) for a medical imaging device comprises: an elongated tubular body having an opening extending along a longitudinal axis (Ax) from a proximal end to a distal end. The elongated tubular body includes a proximal portion (110), a middle portion (120), and a distal portion (130). The proximal portion (110) has a first rigid section (112) and a first flexible section (114). The middle portion (120) has a second rigid section (122); and the distal portion (130) has a second flexible section (132) and a third rigid section 134. The first flexible section (114) is a section of the tubular body contained between the first rigid section (112) and the second rigid section (122), and the second flexible section (132) is arranged along the tubular body proximal to the second rigid section (122). The first rigid section is configured to couple the tubular drive shaft (100) to a torque input apparatus (202), and the tubular drive shaft is configured to transfer a torque from the torque input apparatus to the distal end of the tubular body. The first flexible section 114 is configured to suppress or minimize torsional distortion effects of the torque transmitted from the proximal to the distal portions, and the second flexible section is configured to provide flexibility of the distal portion for improved drive shaft navigation. The first flexible section (114) and the second flexible section can have similar or different flexible structures. In certain embodiment, the flexible structures may include slot cut patterns or coiled wires along different sections of the drive shaft. In other embodiments, the flexible structures may include a combination of slot cut patterns and coiled wires along different sections of the drive shaft.

These and other objects, features, and advantages of the present disclosure will become apparent upon reading the following detailed description of exemplary embodiments of the present disclosure, when taken in conjunction with the appended drawings, and provided claims.

### BRIEF DESCRIPTION OF DRAWINGS

Further objects, features and advantages of the present disclosure will become apparent from the following detailed description when taken in conjunction with the accompanying figures showing illustrative embodiments of the present disclosure.

FIG. 1 illustrates a first exemplary embedment of a flexible tubular drive shaft 100 for a medical imaging device, according to the present disclosure.

FIG. 2A, FIG. 2B, and FIG. 2C illustrate details of a first flexible section 114 of the tubular drive shaft 100.

FIG. 3A, FIG. 3B, and FIG. 3C illustrate details of a second flexible section 132 of the tubular drive shaft 100.

FIG. 4 illustrates a functional diagram of the elements forming the flexible tubular drive shaft 100, according to the present disclosure.

FIG. 5 shows a second embodiment of the tubular drive shaft 100.

FIG. 6 shows a third embodiment of the tubular drive shaft 100.

FIG. 7 shows a fourth embodiment of the tubular drive shaft 100.

FIG. 8 illustrates an exemplary medical device 800 in which the drive shaft 100, according to the various embodiments of present disclosure may be practiced.

### DETAILED DESCRIPTION OF EXEMPLARY EMBODIMENTS

The various embodiments disclosed herein are based on an objective of providing a hybrid flexible drive shaft which comprises a proximal part which has a semi-rigid torque coil and a slot cut metal tube for transmission of torque and a distal part which has a flexible torque coil for navigating tight bending paths to drive rotary imaging devices located at the distal end of an optical probe. A flexible single layer or multiple-layer torque coil is used for the distal part of the tight bend tortuous driving path. A straight metal tube is connected to the torque coil at its distal end and connected with a drive power unit at its proximal end for the segment of the straight drive path. The proximal end of the straight metal tube has slot cuts to provide a flexible coupling between the drive shaft and a motor. At the distal end of the drive shaft, a tube (or metal can) is connected with the flexible torque coil section, the tube (or metal can) serves to support or house therein micro-optic imaging devices of an imaging probe.

Throughout the figures, the same reference numerals and characters, unless otherwise stated, are used to denote like features, elements, components or portions of the illustrated embodiments. In addition, while the subject disclosure is described in detail with reference to the enclosed figures, it is done so in connection with illustrative exemplary embodiments. It is intended that changes and modifications can be made to the described exemplary embodiments without departing from the true scope and spirit of the subject disclosure as defined by the appended claims. Although the drawings represent some possible configurations and approaches, the drawings are not necessarily to scale and certain features may be exaggerated, removed, or partially sectioned to better illustrate and explain certain aspects of the present disclosure. The descriptions set forth herein are not intended to be exhaustive or otherwise limit or restrict the claims to the precise forms and configurations shown in the drawings and disclosed in the following detailed description.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached”, “coupled” or the like to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown in one embodiment can apply to other embodiments. It will also be appreciated by those of skill in the art that references to a structure or feature that is disposed “adjacent” to another feature may have portions that overlap or underlie the adjacent feature.

The terms first, second, third, etc. may be used herein to describe various elements, components, regions, parts and/or sections. It should be understood that these elements, components, regions, parts and/or sections are not limited by these terms of designation. These terms of designation have been used only to distinguish one element, component, region, part, or section from another region, part, or section. Thus, a first element, component, region, part, or section discussed below could be termed a second element, component, region, part, or section merely for purposes of distinction but without limitation and without departing from structural or functional meaning.

As used herein, the singular forms “a”, “an”, and “the” are intended to include the plural forms as well, unless the context clearly indicates otherwise. It should be further understood that the terms “includes” and/or “including”, “comprises” and/or “comprising”, “consists” and/or “consisting” when used in the present specification and claims, specify the presence of stated features, integers, steps, operations, elements, and/or components, but do not preclude the presence or addition of one or more other features, integers, steps, operations, elements, components, and/or groups thereof not explicitly stated. Further, in the present disclosure, the transitional phrase “consisting of” excludes any element, step, or component not specified in the claim. It is further noted that some claims or some features of a claim may be drafted to exclude any optional element; such claims may use exclusive terminology as “solely,” “only” and the like in connection with the recitation of claim elements, or it may use of a “negative” limitation.

The term “about” or “approximately” as used herein means, for example, within 10%, within 5%, or less. In some embodiments, the term “about” may mean within measurement error. In this regard, where described or claimed, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range, if recited herein, is intended to include all sub-ranges subsumed therein. As used herein, the term “substantially” is meant to allow for deviations from the descriptor that do not negatively affect the intended purpose. For example, deviations that are from limitations in measurements, differences within manufacture tolerance, or variations of less than 5% can be considered within the scope of substantially the same. The specified descriptor can be an absolute value (e.g. substantially spherical, substantially perpendicular, substantially concentric, etc.) or a relative term (e.g. substantially similar, substantially the same, etc.).

As it is known in the field of medical devices, the terms “proximal” and “distal” are used with reference to the manipulation of an end of an instrument extending from the user to a surgical or diagnostic site. In this regard, the term “proximal” refers to the portion of the instrument closer to the user, and the term “distal” refers to the portion of the instrument further away from the user and closer to a surgical or diagnostic site. As it is known in the field of medical devices, the terms “proximal” and “distal” are used with reference to the manipulation of an end of an instrument extending from the user to a surgical or diagnostic site. In this regard, the term “proximal” refers to the portion of the instrument closer to the user, and the term “distal” refers to the portion of the instrument further away from the user and closer to a surgical or diagnostic site.

As used herein the term “catheter” generally refers to a flexible and thin tubular instrument made of medical grade material designed to be inserted through a narrow opening into a bodily lumen (e.g., a vessel) to perform a broad range of medical functions. The more specific term “optical catheter” refers to a medical instrument comprising an elongated bundle of one or more flexible light conducting fibers disposed inside a protective sheath made of medical grade material and having an optical imaging function. A particular example of an optical catheter is fiber optic catheter which comprises a sheath, a coil, a protector and an optical probe. In some applications a catheter may include a “guide catheter” which functions similarly to a sheath. As used herein the term “endoscope” refers to a rigid or flexible medical instrument which uses light guided by an optical probe to look inside a body cavity or organ. A medical procedure, in which an endoscope is inserted through a natural opening, is called an endoscopy.

The exemplary embodiments disclosed herein are directed to micron-sized fiber-optic-based imaging probes that can be fabricated easily, at low cost, and can maintain the ability to provide high quality images. As used herein, micron-sized imaging probes and optical elements thereof may refer to components having physical dimensions of 1.5 millimeters (mm) or less in diameter.

### First Embodiment

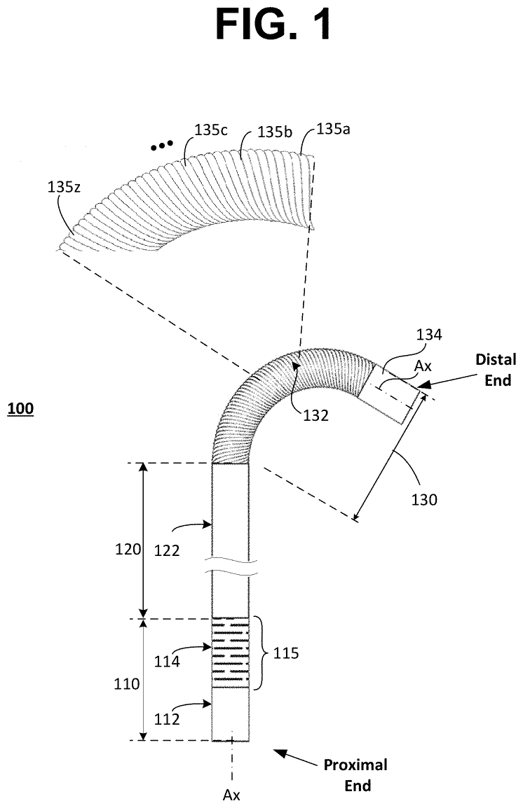

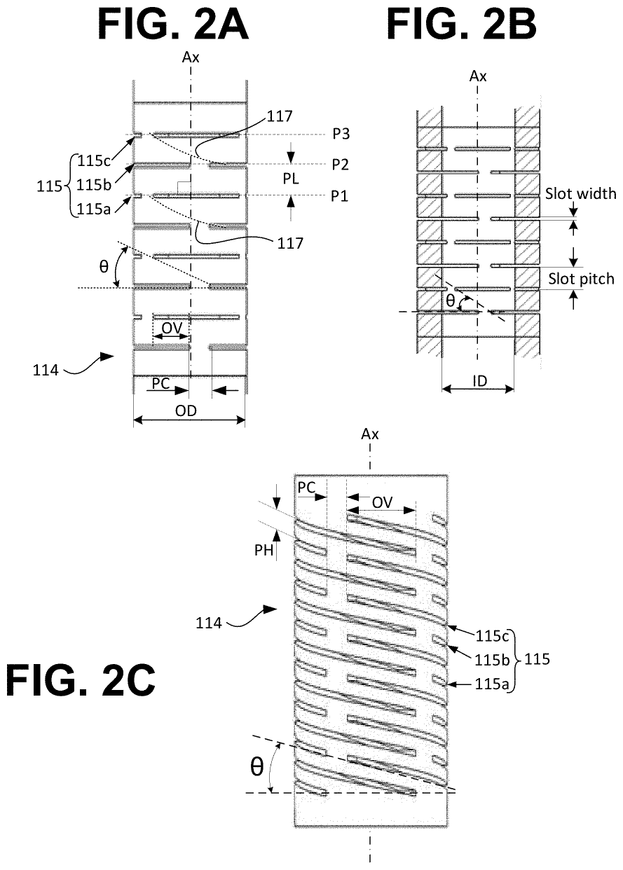

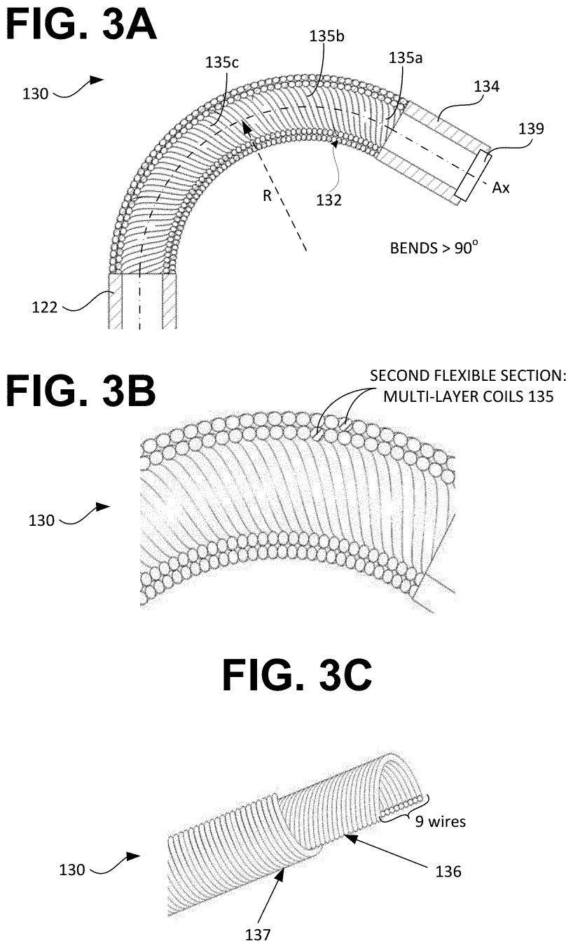

A first exemplary embedment of a novel tubular drive shaft 100 is described in reference to FIG. 1, FIGS. 2A, 2B, 2C, and FIGS. 3A, 3B, and 3C. FIG. 1 illustrates a first exemplary embedment of a flexible drive shaft (100) for a medical imaging device, FIGS. 2A-2C illustrate details of a first flexible section of the tubular drive shaft 100, and FIGS. 3A-3C illustrate details of a second flexible section of the tubular drive shaft 100, according to the present disclosure.

As shown in FIG. 1, a tubular drive shaft (100) for a medical imaging device comprises an elongated tubular body having an opening extending along a longitudinal axis (Ax) from a proximal end and a distal end. The elongated tubular body includes a proximal portion 110, a middle (center) portion 120, and a distal portion 130. The proximal portion 110 has a first rigid section 112 and a first flexible section 114. The middle portion 120 has (or is) a second rigid section 122; and the distal portion 130 has a second flexible section 132 and a third rigid section 134. At least the proximal portion 110 and the middle portion 120 (i.e., the first rigid section 112, first flexible 114, and second rigid 122) can be part a single monolithic metal tube made of medical grade material. For example, the metal tube may be made from a resilient material, such as nickel-titanium (NiTi) alloy or Nitinol, stainless steel, platinum, copper, or other hard metal and composite materials. In some embodiments, at least part of the drive shaft 100 (e.g., the proximal portion 110 and the middle portion 120) can be made of a polymer tube structure where the first flexible section 114 can be reinforced with metallic wire built-in within the wall of the tube structure, such as a hypotube. The distal portion 130 includes the second flexible section 132 and the third rigid section 124. The second flexible section 132 includes a plurality of wire coils (135a, 135b, 135c . . . , 135z) as shown in the expanded inset on the upper left-hand side of FIG. 1.

The third rigid section 134 is for housing imaging micro-optics such as focusing and dispersive optical components, e.g., a spacer, a ball lens, GRINS lens, etc., which enclosed in a short piece of metallic tube called “CAN” in SEE (spectrally encoded endoscopy). In the present disclosure, some embodiments, the distal end rigid section may be a “rigid tube” configured to enclose distal optics. However, in some embodiments, the distal optics may be enclosed in the distal end of the second flexible section. In that case, the third rigid section is not used. In other embodiments, the third rigid section is a rigid tubular part with different designs to accommodate the distal end components of a given modality. For example, in an IVUS modality, the third rigid section may include a metal tube configured to house therein one or more transducers and other microelectronic elements related thereto.

In the proximal portion 110, the proximal end of the straight metal tube has a first part (first rigid section 112) that connects to the rotating element of a non-illustrated motor (drive power unit), and a second part (first flexible section 114) that has slot cuts to provide flexible coupling with the drive power unit but inflexible torsionally to transmit the rotational torque with minimal NURD. The length of this segment having slot cuts (i.e., the length of the first flexible section 114) is preferably in the range from about 1 mm to 30 mm (3 centimeter) depending on the application of the medical device. The width of the slots is preferably in a range from about 0.01 mm to about 1 mm.

The first flexible section 114 works as a flexible “coupling” section to connect the two rigid sections (the first rigid section 110 and the second rigid section 122) of the drive shaft. The purpose of this coupling is for transmitting torque between two rigid sections of the shaft while allowing for some degree of accommodation, e.g., allowing small misalignment (angular and parallel offset). In this manner, the first flexible section 114 also serves to minimize tension, e.g., during the process of attaching the drive shaft 100 to the torque input apparatus, by accommodating possible misalignment.

The first flexible section 114 is a section of the tubular body contained between the first rigid section 112 and the second rigid section 122. The proximal portion 110 and middle portion 120 (i.e., the first rigid section 112, first flexible 114, and second rigid 122) can be part a single monolithic metal tube in which the slot cuts are made in a short longitudinal section of the tube between the first rigid section 112 and the second rigid section 122. Using a single monolithic tube allows forming the first flexible section 114 integrally with the other two rigid sections 112 and 122. In an alternative embodiment, the first flexible section 114 can be a separate tubular part proximally connected (e.g., welded) with the first rigid section 112 and distally connected (e.g., welded) with the second rigid section 122. In the case where the first flexible section 114 is a separate component, the first flexible section 114 can have the same diameter or a different diameter than that of the first rigid section 112 and second rigid section 122, and it can be welded or otherwise attached in a manner known to persons of ordinary skill in the art. To provide the necessary flexibility, the first flexible section 114 includes a plurality of slots 115 (115a, 115b, 115c). The slots 115 are cut into the wall of the tubular body and are arranged in a staggered manner so as to be offset from each other around the circumference of the tubular body in a direction of the longitudinal axis.

More specifically, FIG. 2A, 2B, and 2C shows an expanded view of the first flexible section 114. In the expanded view of FIG. 2A, the first flexible section 114 shows a first slot 115a, a second slot 115b, a third slot 115c, etc., which are circular (or arc) cuts made on the wall of the tubular shaft, where the cuts are substantially perpendicular to the longitudinal axis Ax. There may be one or more cuts (slots 115) made through the wall at each at each plane. For example, one or more slot may be cut at a first plane P1, a similar number of slots may be cut at a second plane P2 and at a second plane P3. Planes P1 and P3 are at a longitudinal pitch PL. The slots 115 may be cut at each plane at a predetermined circular distance or circular pitch PC. The slots 115 are offset from each other in a lengthwise direction of proximal end to distal end such that consecutive slots 115 overlap each other a distance (OV) which is about half the length of the slot cut, and ends of the slots 115a, 115b, 115c form a helical or colloidal locus 117 around the wall of the tubular body. As shown in FIG. 2A, the ends of consecutive slot cuts are offset from each other around the surface of the tubular shaft so as to form a locus 117 having a helix angle θ, which may be, for example, in a range from about 1 to 60 degrees, or more preferably greater than zero (0) to equal to or less than 45 degrees (i.e., 0<θ≤45 [degrees]). FIG. 2B shows a cross-sectional view of the first flexible section 114. As shown in FIG. 2B, the slot cuts of the first flexible section 114 are arranged at a slot pitch of about 0.2 mm to 5.0 mm. That is, the slot cuts are arranged in a lengthwise direction at a distance or longitudinal pitch PL of about 0.2 to 5.0 mm. A similar or proportional circular pitch PC may also be used. Moreover, an average width of the slot cuts is in a range from about 0.01 mm to about 1.0 mm.

Many different slot cut patterns can be used to cut a metal tube to make it a flexible shaft, but for torque transition imaging components, the slot cut patterns should be optimized for high torsional inflexibility and easy bending flexibility under the required torque. The expanded view of the first flexible section 114 shown in FIG. 2A and FIG. 2B is an example of a slot cut pattern with good rotational torque transmission performance which results in minimized torsional distortion effects (e.g., low NURD errors). FIG. 2C shows another example of a slot cut pattern for the first flexible section 114. In the example of FIG. 2C, the slots 115 are cut at an angle θ with respect to the normal to the longitudinal axis Ax. The angle θ of the slots is preferably in a range from greater than 0 to equal to or less than 45 degrees (i.e., 0<θ≤45 [degrees]). Similar to the slots of FIG. 2A, the angular slot cuts of the first flexible section 114 shown in FIG. 2C can be arranged at a slot pitch (helical pitch PH) of about 0.2 mm to 5.0 mm. That is, the slot cuts can be angular cuts of about 0.01 mm to 1.0 mm in with made along the wall of the tubular shaft at a distance of about 0.2 to 5.0 mm. Moreover, the distance at which the cuts are made can be a fixed pitch or a variable pitch of about 0.2 to 5.0 mm. The slots can be cut using computer controlled cutting techniques such as laser cutting, Electrical Discharge Machining (EDM), water jet cutting, milling or other similar techniques.

The proximal section 110 of the straight metal tube has the slot cuts to provide a flexible coupling means with the drive power unit, but it is inflexible torsionally to transmit the rotation (torque) with minimal NURD based on the same principle as the flexible motor coupler for motor coupling. For certain applications, the length of this cut segment (i.e., the length of the first flexible section 114) is in a range from about 2 mm to 3 centimeters, and the width of the slots is in a range from about 0.1 mm to about 1 mm.

FIGS. 3A, 3B, 3C illustrate expanded views of the second flexible section 132. More specifically, FIG. 3A shows an expanded view of the distal portion 130; FIG. 3B shows an expanded view of the second flexible section 132; and FIG. 3C shows a view of the second flexible section 132 made of multi-layer coils. In the distal portion 130, the second flexible section 132 includes a plurality of coils 135 which are arranged at the distal end of the middle portion 120. As shown in FIG. 3A to prolong (or to form) the wall of the tubular body, a plurality of coils 135a, 135b, 135c, etc. is connected (or otherwise attached) to the distal end of the second rigid section 122. This second flexible section 132 is a section of the tubular body contained between the second rigid section 122 and the third rigid section 134. The second flexible section 132 is preferably formed separately and connected (welded) to the distal end of the second rigid section 122 and to the proximal end of the third rigid section 134. As shown in the upper left-hand section of FIG. 1, an expanded view of the second flexible section 132 shows a first coil 135a, a second coil 135b, a third coil 153c, etc., which are made of round metal wire tightly wound to form a flexible tubular section connected to the distal end of the middle portion 120. In alternative embodiments, the flexible torque coils can also be made by wound square or rectangular wires. Examples of applicable materials for the coil wire include, but are not limited to, Nitinol, Stainless still, Titanium, Nickel, Copper, and other like materials and alloys thereof.

FIG. 3A, FIG. 3B to FIG. 3C show expanded views of the second flexible portion 132. The second flexible section 132 can be comprised of a single layer of tightly wound wires or of a multiple-layer of wire coils. FIGS. 3A and 3B show the second flexible section 132 is formed of two layers of wire coils arranged substantially concentric with the longitudinal axis Ax. As shown in FIG. 3A, the second flexible portion 132 connects to the distal end of the second rigid portion 122 and to the proximal end of the third rigid section 134. The second flexible portion 132 is made of generally round or circular cross-section wires which are tightly wound together, and configured to create a bent curve of at least 45 degrees, and more preferably a bending curve of 90 degrees or more (bend 90 [degrees]).

FIG. 3C shows an example of a multiple-layer coil where each layer is comprised of multiple wires or filars tightly wound adjacent to each other. FIG. 3C shows a two-layer torque coil having an inner layer 136 and an outer layer 137. The inner layer 136 has nine filars or wires. For the multiple-layer coil, the adjacent layers (the first layer and second layer) are usually wound in opposite directions to further improve torque transmission performance and reduce NURD. The second flexible section 132 may also be referred as the “torque coil section” where the torque coil is designed to be flexible (more flexible than the first flexible section 114) for navigating through tight bending paths.

In most embodiments, the distal portion 130 of the tubular shaft is more flexible than the proximal portion 110, and the middle portion is generally made of a rigid metal tube. To make the distal portion 130 more flexible than the proximal portion 110, in most embodiments, the first flexible portion 114 is made of a slot cut pattern and the second flexible portion 132 is made of tightly wound coil. In certain embodiments, however, the distal portion 130 may be equally flexible or equally rigid as the proximal portion. In that case, both the first flexible portion 114 and the second flexible portion 132 can be made of the same flexible structure. That is, in some embodiments, both the first flexible portion 114 and the second flexible portion 132 can be made of a slot cut pattern or of tightly wound coil. Moreover, even in those embodiments where the distal portion 130 is more flexible than the proximal portion 110, both the first flexible section 114 and the second flexible section 132 can be made of the same flexible structure (e.g., a slot cut pattern or coiled wire pattern), but the different levels of flexibility can be provided by the specific manner in which the respective flexible structure is formed. For example, in certain embodiments it would be advantageous if both flexible sections are made of slotted flex drive cables. The different levels of flexibility in the first flexible section 114 and the second flexible section 132 can be provided by varying the slot cut pattern, slot size, slot pitch, etc. A drive shaft having this structure would be much easier to make (e.g., in terms of manufacturing cost and time) than making a drive shaft with hybrid flexible structures where coils and slotted tube structure are combined and welded together. In certain embodiments, however, a drive shaft having a hybrid flexible structure may be more desirable in order to optimize torque transmission, torsion inflexibility, and bending flexibility, which minimizing negative torsional effects such as buckling and NURD.

In one aspect of the present disclosure, the semi-rigid tubular shaft is a catheter having a distal portion with a reduced rigid length or a distal portion which is not rigid at all. The distal portion having a reduced rigid length can allow the catheters to access and treat tortuous vessels and small diameter bodily lumens. In most embodiments, a rigid distal portion or housing of the tubular shaft has a diameter that generally matches the diameter of the proximal portion of a catheter body. However, in other embodiments, the distal portion may have a diameter larger or smaller than that of the flexible portion. Additionally, some embodiments include a flexible distal tip without the rigid housing (or metal can).

### <Functional Block Diagram>

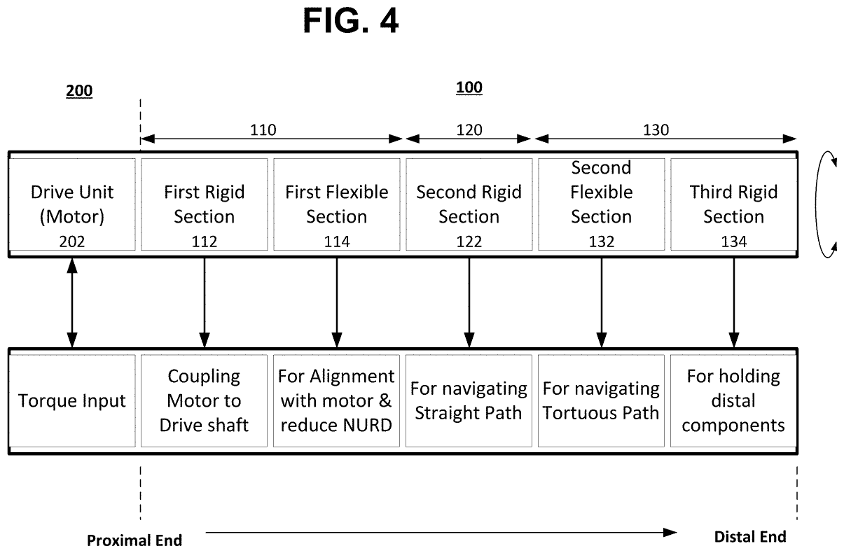

FIG. 4 is a block diagram showing the functionality of each section of the tubular drive shaft 100. As shown in FIG. 2, the functionality of each section of the tubular drive shaft 100 is described as follows. At the proximal end of the tubular drive shaft 100, a drive unit (motor) 202 serves as the torque input apparatus provided to the tubular body of the tubular drive shaft 100. Since the tubular drive shaft 100 is designed to have a long rigid section 122 with at least one flexible section in the proximal portion 110 and at lest one flexible section in the distal portion 130, the tubular drive shaft 100 of the present disclosure is configured to transfer the torque from the drive unit 202 to the distal end with minimal NURD effect. To that end, the first rigid section 112 serves as a mechanical coupling to the drive unit 202 (motor) arranged at the proximal end of the drive shaft. The first rigid section 112 is a short metal tube sufficiently resilient to couple the motor to the tubular drive shaft 100. The first flexible portion 114 includes a short segment of straight metal tube with slot cuts made along the wall of the tube. As noted above, the slot cuts can be made perpendicular or slated at an angle with respect to the longitudinal axis. The first flexible portion 114 provides a level of flexibility and resilience to correct possible errors caused by misalignment of the tubular drive shaft 100 with the drive unit (motor) 202. Specifically, in the case where the tubular drive shaft 100 has even a small amount of misalignment with the motor of the drive unit 202, the high speed rotation of the motor tends to cause vibration and wobbling of the tubular drive shaft 100. This vibration or wobbling can be detrimental to delicate anatomies of a patient, or to the imaging components arranged at the distal end of the probe, and/or to the imaging quality. To address these issues, the inventor herein has realized that a short flexible structure (e.g., the slotted cuts) made in the proximal section (first flexible portion 114) of the drive shaft effectively avoids vibration and wobbling in the distal section of the tubular drive shaft. In other words, the proximal portion 110 of the tubular drive shaft 100 works as a semi-flexible motor shaft coupler.

The middle portion 120 of the tubular drive shaft 100 is the second (and longest) rigid section 122 of the drive shaft. Preferably, the second rigid section 122 is a single layer of straight metal tube having a length larger than the proximal portion 110 and larger than the distal portion 130. The straight metal tube is used for the segment of a straight drive path. The second rigid section 122 is connected to the second flexible section 132 (torque coil section) at its distal end and connected with the first flexible section 114 and the drive power unit at its proximal end. Preferably, the metal tube (proximal portion 110 and middle portion 120) has the same outer diameter (OD) as the torque coil section and as the first flexible section. Welding processes, such as laser welding, ultrasonic welding, soldering, brazing, or other bonding techniques can be used to connect the metal tube of the middle portion 120 with the flexible sections (metal torque coil) in the distal portion 130 and the proximal portion 110. The rigid metal tube of the middle portion 120 is effective in transferring the torque force from the proximal portion 110 to the distal portion 130, which reducing NURD errors.

At the distal portion 130, the second flexible section 132 (the torque coil section) is used for navigating tortuous paths of tight bending and delicate structures. In some applications, see FIG. 3A, a minimum bending radius R of the driving path is preferably in, but not limited to, a range from about 2 mm to 50 mm. To effectively navigate through tight ending anatomical paths, it is preferable that second flexible section 132 provides enough flexibility to bend the distal portion 130 by an angle of at least 90 degrees.

Further, at the distal portion 130, the third rigid section 134 serves as a housing or enclosure for holding imaging components, such as the distal optics of an imaging catheter. For the third rigid section 134, another relatively short metal tube is connected to the torque coil section at the distal end thereof to support or host the imaging devices. Preferably, the metal tube of the third rigid section 134 has the same OD as the torque coil section. As shown in FIG. 3A, the third rigid section 134 may include a transparent window 139 to allow transmission of light therethrough for forward view imaging. Similar to the other sections, welding (laser weld or ultrasonic weld) or other bonding techniques can be used to connect the metal tube of the third rigid section 134 to the coils of the second flexible section 132, and to the window 139.

Modification of first embodiment. The structure of the tubular drive shaft 100 shown in FIG. 1 can be modified such that the first rigid section 112, second rigid section 122 and the third rigid section 134 are not formed from a metallic tube of a same diameter. Instead, the straight rigid sections 112, 122 and 134 on both ends of the two flexible sections 114, 132 can be made of non-metal tubes. For example, one or more of the first rigid section 112, the second rigid section 122, and the third rigid section 134 can be made of rigid polymer material bonded (or otherwise attached) to the flexible section by, for example, ultrasound welding or boding adhesives. Examples of polymer materials applicable for the rigid sections 112, 122, and 134 include, but are not limited to, ABS (Acrylonitrile-Butadiene-Styrene), Nylon, PC (polycarbonate), PEEK (Polyetheretherketone), PET (Polyethylene Terephthalate), PI (Polyimide), and the like.

In addition, as a further modification, the tubular drive shaft 100 can be provided with the distal portion 130 having a different diameter than the diameter of the proximal portion 110 and middle portion 120. For example, the second flexible section 132, and the third rigid section 134 (i.e., the torque coil section and the metal tube at the distal end of the torque coil section) may have an outer diameter (OD2) smaller than an outer diameter (OD1) of the first rigid section 112, the first flexible section 114, and the second rigid section 122. Advantageously, having a distal portion 130 of a smaller dimeter than the proximal portion 110 and middle portion 120 (i.e., OD2<OD1) allows the tubular drive shaft 100 to go through or go inside small anatomies while keeping the dimensions and strength of the proximal sections for accurate coupling and minimal NURD effect. In other embodiments, the middle portion 120 and the distal portion 130 may have an outer diameter (OD) smaller than the OD of the proximal portion 110. The smaller OD distal section allows the drive shaft to go through or go inside small anatomies while the proximal end with a larger OD, which is usually outside of the patient's anatomy or body, has the appropriate dimensions and strength to couple/adapt with the rotary source/motor.

### Second Embodiment

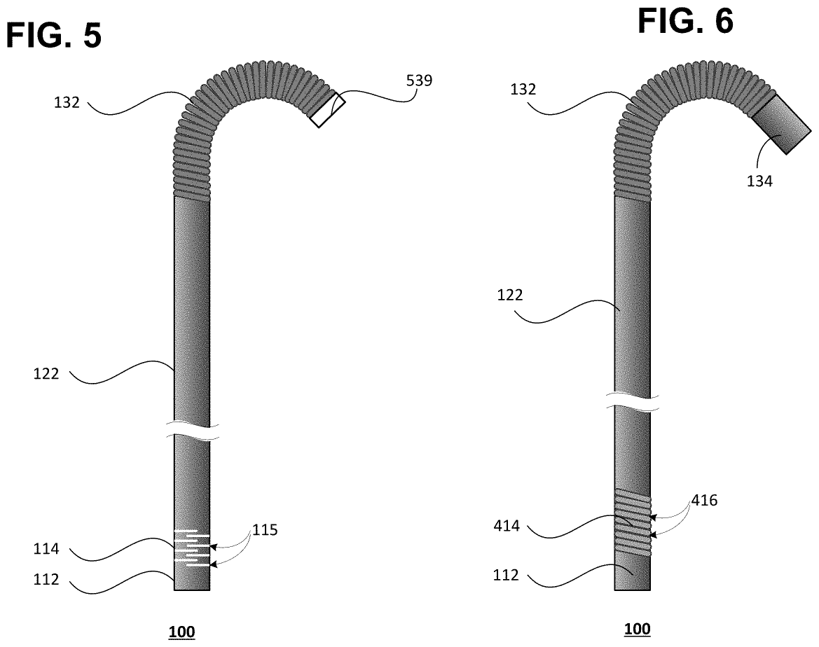

FIG. 5 shows a second embodiment of the tubular drive shaft 100. The embodiment shown in FIG. 5 is substantially similar to the embodiment shown in FIG. 1-FIG. 3C. In this second embodiment, the third rigid section 134 is omitted. That is, the tubular drive shaft 100 according to the second embodiment does not have the metal tube at the distal end of the drive shaft. In this case, the imaging components at the distal end of the tubular drive shaft 100 are bonded directly to the distal end of the second flexible section 132 (torque coil section). The embodiment shown in FIG. 5 can be applicable, for example, to forward-view imaging probes where the illumination light and collected light can be transmitted through a transparent window (e.g., glass window) 539 arranged at the distal tip of the second section 132 (torque coil section).

### Third Embodiment

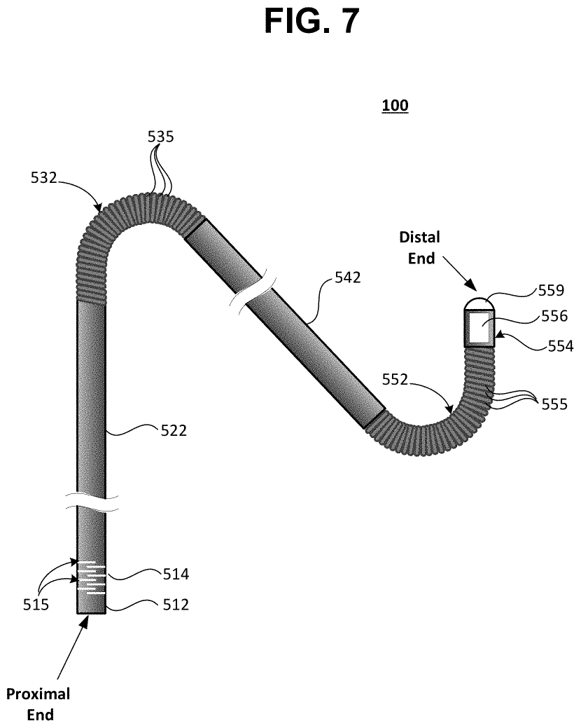

FIG. 6 shows a third embodiment of the tubular drive shaft 100. The embodiment shown in FIG. 6 is substantially similar to the embodiment shown in FIG. 1-FIG. 3C. In this embodiment, the first flexible section 114 is modified. Specifically, in the embodiment of FIG. 6, the straight metal tube having slotted cuts 115 is replaced by a same length of torque coil 414 having a plurality of coils 416. The design of the torque coil 414 can be the same as the torque coil section of the second flexible portion 132. That is, in FIG. 6, the first flexible section 114 may include a single layer or multi-layer coil similar to the flexible structure of the second flexible section 132, where the coil is formed by tightly wound wire, where the wire is round circular cross-section wire or rectangular cross-section wire. In addition, as discussed above, the first flexible section 114 and the second flexible section 132 may both include cut slots 115 with similar (or different) patterns, sized, pitch, etc. In other words, according to the embodiment of FIG. 6, the flexible structure of the first flexible section 114 and the second flexible section 132 can be the same, but to provide the different degrees of flexibility, at least one parameter of the flexible structure is different. In this case, even if the same type of coil or slot cuts are used in the flexible structure, the length of the first flexible section 114 can be shorter than the length of the second flexible section 132, so that the first flexible section 114 becomes less flexible than the second flexible section 132. The embodiment shown in FIG. 6 can be applicable, for example, to either forward-view or side-view imaging probes. In the case of side-view imaging, the third rigid section or metal can 134 of the flexible drive shaft of FIG. 6 can have a transparent window and an atraumatic distal tip (cap) filled with radiopaque material, similar to that shown in FIG. 7.

### Fourth Embodiment

FIG. 7 shows a fourth embodiment of the tubular drive shaft 100. According to FIG. 7, the tubular drive shaft 100 includes a proximal portion 110 with a flexible section, and a distal portion 130 with a flexible section, similar to that of first, second and third embodiments. However, the middle portion 120 is modified to include more than one straight rigid tube for straight drive path and more than one flexible segment of torque coils for bent path or articulated operations. Specifically, as illustrated in FIG. 7, the tubular drive shaft 100 according to the forth embodiment includes, in order from the proximal end to the distal end, a first rigid section 512, a first flexible section 514 having a plurality of slots 515 cut into the wall of the tubular body, a second rigid section 522, a second flexible section 532, a third rigid section 542, a third flexible section 552, a fourth rigid section 554. In this embodiment, the first flexible section 514 can also be modified such that the slot cuts (slots 515) on the straight metal tube are replaced by a same length of torque coil section. Alternatively, all flexible sections can be made of slots 515 cut into the wall of the drive shaft. Making all flexible sections of slot cuts would allow the drive shaft to be formed of a single tubular structure without requiring welding or other type of mechanical attachment of the different sections of the drive shaft. The fourth rigid section 554 can have a transparent window 556 for side-view imaging and an atraumatic distal tip (cap) 559 filled with radiopaque material. The fourth rigid section 554 may be made from a metal tube similar to the first and second rigid sections described with reference to FIG. 1, for example, nitinol or stainless steel; and the distal end of the metal tube may be filled with a substantially radiopaque-material such as platinum or tungsten. One advantage of having more than one straight rigid tubes (more than one rigid section) and more than one flexible segment (e.g., of torque coils) is to make the distal side sections of the first torque coil 532 manipulatable or bendable relative to the straight metal tube of second rigid section 522. This provides the freedom for the distal sections to be bent or articulated to different directions and moved to different locations. Again, the minimized total torque coil length, in the proximal portion of the drive shaft, is used to minimize the NURD effect from the flexible torque coils.

According to the various embodiments disclosed herein, a semi-rigid drive shaft 100 for an imaging device comprises a proximal portion having a first flexible section, a middle portion having a rigid section made of a straight metal tube, and a distal portion having a second flexible section. The straight metal tube is used for the segment of the straight drive path. The first and/or second flexible sections consist of a single layer or multiple-layer torque coil. In one embodiment, the first flexible section is a short segment of a metallic tube with slot cuts. The second flexible section is a torque coil is used for navigating tight bend tortuous paths. In some, but not all, embodiments, the distal portion includes a short tube (or metal can) at the distal end of the second flexible section to support or host the imaging optics of a probe. The slot cuts of the first flexible section at proximal end of the straight tube provide flexible coupling and alignment of drive power unit to the middle portion having a rigid section made of a straight metal tube.

The first flexible section must have a certain degree of rigidity and also be flexible to minimize NURD. A balance between rigidity and flexibility can be achieved through the design of the slot pattern. Many different slot cut patterns can used to cut metal tube to make it flexible, but for torque transition imaging components, the slot cut pattern should be optimized for minimal NURD under the required torque and good flexibility. FIG. 1 shows one example of slot cut pattern, but other patterns are considered to be within the knowledge of persons having ordinary skill in the art. The slots can be cut using computer controlled cutting techniques such as laser cutting, water jet cutting, milling or the like. As an example, some of techniques and micro-cutting systems for forming cuts in catheters, guidewires, and similar products, as disclosed in U.S. Pat. Nos. 10,232,141 and 5,741,429, can be used for making the slot cuts in the metal tube. Further, different degrees of flexibility of the tubular shaft can be achieved by having different width and/or different pitch, and/or different patterns of slots, and/or different lengths of the tube where slots are formed. A width of the slots is in a range from about 0.01 mm to about 1.0 mm, and a pitch of slots in range of about 0.2 to 5 mm was found to be acceptable for certain applications. However, the length and width of the slots too can be determined experimentally to provide the required flexibility and minimized NURD according to the desired application.

Some advantages provided by the drive shaft described in the present disclosure include slot cuts at proximal end of an straight tube to provide flexible coupling and alignment of the drive shaft with drive power unit; minimized torque coil length at the distal end to reduce imaging NURD and to drive through tortuous or tight bend paths; straight rigid tube in the middle portion of drive shaft to transmit rotational torque from the torque input apparatus to the straight drive path without (or with minimal) NURD effect. According to the present disclosure, the tubular drive shaft comprises a straight rigid tube with a short segment of slot cuts near the proximal end, a long and rigid middle portion, a single layer or multiple-layer torque coil, and a short tube (or metal can) at the torque coil's distal end. The torque coil is used for the portion of tight bend in tortuous drive paths. The rigid straight tube (metal or polymer) is used for the segment of the straight drive path. The straight tube is connected to the torque coil at its distal end and connected with the drive power unit at its proximal end. The proximal end of the straight tube has slot cuts to provide a flexible coupling and resilient torsion in the link with the drive power unit. A relatively short tube (metal can) is connected to the torque coil at the distal end of the straight tube to support or host imaging devices or other elements.

The present disclosure is generally directed a hollow flexible tubular drive shaft applicable to a medical device. Medical devices that use this type of flexible tubular drive shafts usually have a sheath outside of the shaft and other functioning components inside. The specific applications of the novel drive shaft are not the focus of the present disclosure. Nevertheless, some embodiments make reference optical imaging and optical components in order to give context to exemplary applications of the flexible tubular drive shaft. Those of ordinary skill in the art will appreciate that the present disclosure is applicable to other applications such as IVUS which uses sound waves, and OCT imaging which uses low-coherent light. Indeed, some applications beyond endoscopic imaging may include, for example, other medical devices such as surgical drilling, biopsy needle guidance, and the like.

Intravascular ultrasound (IVUS) is a catheter-based medical imaging methodology using a specially designed catheter with a miniaturized ultrasound probe attached to the distal end of the catheter. The proximal end of the catheter is attached to computerized ultrasound equipment and the distal end is introduced and guided through blood vessels. IVUS allows the application of ultrasound technology to see from inside blood vessels out through the surrounding blood column, visualizing the endothelium (inner wall) of blood vessels in living individuals. IVUS catheters are generally designed for use as an adjunct to conventional angiographic procedures to provide an image of the vessel lumen and wall structures. Optical coherence tomography (OCT) is an imaging technique that uses low-coherence light to capture micrometer-resolution, two- and three-dimensional images from within optical scattering media (e.g., biological tissue or vessel wall). In either IVUS or OCT, the tubular shaft 110 may be a torque shaft that facilitates passage and navigation of a catheter body to a diseased or examination site. The proximal end of the torque shaft is coupled to a handle and the distal end of the torque shaft is attached to the distal, rigid portion of the catheter through a connection assembly.

### <Drive Shaft Use in Medical Imaging Applications>

Having described various embodiments of the disclosure in detail, it is instructive to present an example environment in which the disclosed embodiments of may be implemented. According to one example, the tubular drive shaft 100 can be used as a drive shaft for driving imaging components of a catheter-based intraluminal OCT system using a proximal control mechanism.

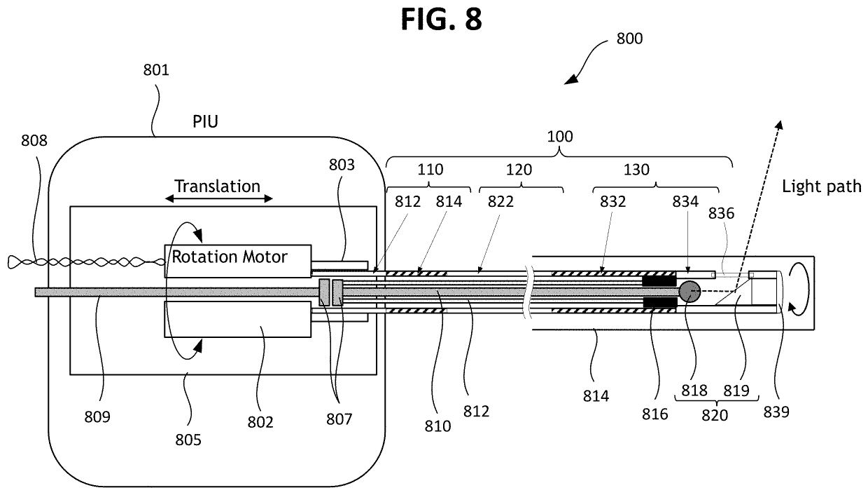

FIG. 8 illustrates an example embodiment of a catheter-based medical device 800 having a proximal control mechanism. As illustrated in FIG. 8, a proximal control mechanism includes a patient interface unit (PIU) 801 connected to the proximal end of the drive shaft 100. Among other elements, the PIU 801 includes a hollow core rotational motor 802 (proximal torque motor) and a pullback translation stage 805 (pullback motor), both located inside the patient interface unit 801. The rotational motor 802 is equivalent to the drive unit (motor) 202 shown in FIG. 4. The working principles of a translation stage and a rotational motor having a hollow core are well known to persons of ordinary skill in the art. For this exemplary embodiment, the hollow motor 802 has an inner diameter larger than an outer diameter of a light-guiding component 810 (e.g., an optical fiber or a fiber bundle). The hollow motor 802 is configured to rotatably drive the drive shaft 100 with, for example, a threaded hollow tube 803. The hollow core rotational motor 802 and drive shaft 100 are used to rotate at least part of a distal optics assembly 820 (distal optical system). More specifically, the rotational motor 802 rotates the drive shaft 100; and the drive shaft 100 delivers the torque to the distal optics assembly 820. The distal optics assembly may include, but is not limited to, a focusing component 818 (e.g., a ball lens or GRIN lens) and a reflective and/or diffusing component 819 (e.g., one or more of a mirror, a prism, a grating, etc.). The light-guiding component 810 is arranged inside the drive shaft 100. Depending on design choice or on the specific application, the light-guiding component 810 may be rotatable or non-rotatable. In the example embodiment of FIG. 8, the light-guiding component 810 includes an optical fiber which stays non-rotational; the fiber can go through the central hole (bore) of the motor 802. A stationary fiber connector 807 is used to connect the fiber 810 to a light source fiber 809, and electrical wiring 808 is used to connect the rotational and translation motors to detection and control electronics (not shown). An outer sheath 814 covers at least partially the rotating drive shaft 100. An inner or protective sheath, jacket, or tubing 812 covers the stationary fiber 810. Both the outer sheath 814 and inner sheath 816 stay non-rotational while the drive shaft rotates and transfers torque to at least part of the distal optics assembly 820. The rotational motor 802 and the fiber 102 can be mounted on a linearly motor 805 that performs pullback under programmed control. Pullback can also be performed by a linear translation stage, e.g., using piezoelectric and/or MEMS actuators instead of a motor. In this manner, the catheter including both rotational and non-rotational components can be simultaneously rotated at least 360 degrees and translated longitudinally during pullback to image a target sample (not shown) through a side-view imaging window 836. Mechanical micro bearings 816 can be placed between the rotational and non-rotational (stationary) components of the catheter-based imagining system to reduce friction.

Moreover, in accordance with principles of the present disclosure, the drive shaft 100 includes a proximal portion 110 having a first rigid section 812 and a first flexible section 814; a middle portion 120 having a second rigid section 822; and a distal portion 130 having a second flexible section 832 and a third rigid section 834. The first rigid section 812 is configured to couple the tubular drive shaft 100 to the rotational motor 802 (a torque input apparatus). The first flexible section 814 is configured to slightly bend a section of the drive shaft 100 between the first rigid section 812 and the second rigid section 822. As noted elsewhere in this disclosure, a primary purpose of the first flexible section 814 is for improving the coupling of torque from the torque input apparatus to the remainder of the drive shaft. The purpose of improving coupling is for transmitting torque between the two sections of the shaft while allowing for some degree of misalignment (angular and parallel offset), in particular during the initial process of engaging the drive shaft to the PIU 801. The first flexible section 114 can provide some bending which can help reduce tension, wobble, and/or vibration in the long semi-rigid drive shaft during rotation. The second flexible section 832 is configured to bend a distal section of the drive shaft 100, and to further improve fidelity in the transmission of torque to the third rigid section 834 with is a metallic tube enclosing rotatable distal optics. The second flexible section and the rotatable third rigid section are arranged along the tubular body of the drive shaft 100 distally to the second rigid section 822. The configuration of the example illustrated in FIG. 8 allows for easy connecting and disconnecting of the drive shaft 100 from the proximal control mechanism (PIU), which can be advantageous when using a disposable imaging catheter.

In referring to the description, specific details are set forth in order to provide a thorough understanding of the examples disclosed. In other instances, well-known methods, procedures, components and circuits have not been described in detail as not to unnecessarily lengthen the present disclosure. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

1. A tubular drive shaft for a medical device, comprising:
a tubular body having an outer surface and an inner surface defining a wall that encloses a substantially circular opening that extends along a longitudinal axis from a proximal end to a distal end;
the tubular body having more than one flexible section and more than one rigid section, including:
a proximal portion having a first rigid section and a first flexible section;
a middle portion having a second rigid section; and
a distal portion having a second flexible section,
wherein the first rigid section is configured to couple the tubular drive shaft to a torque input apparatus, and the tubular drive shaft is configured to transfer a torque from the torque input apparatus to the distal end of the tubular body,
wherein the first flexible section is configured to accommodate alignment between the tubular body and the torque input apparatus, and the second flexible section is configured to bend the distal portion of the tubular body, and
wherein the first flexible section is less flexible than the second flexible section.

2. The tubular drive shaft according to claim 1, further comprising:
a third rigid section arranged along the tubular body distally to the second flexible section.

3. The tubular drive shaft according to claim 2,
wherein the second flexible section includes a plurality of coils which are arranged along the tubular body distally to the second rigid section,
wherein the third rigid section is a metallic tube attached to one or more coils at the distal end of the second flexible section, and
wherein a distal end of the metallic tube includes a transparent window for forward view imaging.

4. The tubular drive shaft according to claim 2,
wherein the second flexible section includes a plurality of coils which are arranged along the tubular body distally to the second rigid section,
wherein the third rigid section is a metallic tube attached to one or more coils at the distal end of the second flexible section,
wherein the metallic tube includes a transparent window for side view imaging,
wherein the distal end of the metallic tube is filled with radiopaque material, and the radiopaque material is shaped as an atraumatic cap.

5. The tubular drive shaft according to claim 1,
wherein the first flexible section includes a plurality of slots which are arranged in a staggered manner along the wall of the tubular body, and
wherein the second flexible section includes a plurality of coils which are arranged along the tubular body distally to the second rigid section.

6. The tubular drive shaft according to claim 5,
wherein at least some of the slots among the plurality of slots in the first flexible section are circular slots cut through the wall of the tubular body, and
wherein the circular slots are aligned parallel to a plane that is perpendicular to the longitudinal axis of the tubular body, and
wherein the circular slots are arranged staggered around the wall of the tubular body such that ends of the circular slots form a helical locus, and wherein the helical locus has an angle θ with respect to the plane perpendicular to the longitudinal axis of the tubular body, where 0<θ≤45 degrees.

7. The tubular drive shaft according to claim 6, wherein the circular slots are laser-cut slots arranged at a lengthwise distance of about 0.2 mm to 5.0 mm.

8. The tubular drive shaft according to claim 6, wherein an average slot width of the circular slots is in a range from about 0.1 mm to about 1.0 mm.

9. The tubular drive shaft according to claim 5,
wherein at least some of the slots among the plurality of slots in the first flexible section are helical slots cut through the wall of the tubular body, and
wherein the helical slots are cut at an angle θ with respect to the plane perpendicular to the longitudinal axis of the tubular body, where 0<θ≤45 degrees.

10. The tubular drive shaft according to claim 9, wherein the helical slots are laser-cut slots arranged at a helical pitch of about 0.2 mm to 5.0 mm.

11. The tubular drive shaft according to claim 9, wherein an average slot width of the helical slots is in a range from about 0.1 mm to about 1.0 mm.

12. The tubular drive shaft according to claim 1,
wherein the first flexible section includes a plurality of coils contained in the tubular body between the first rigid section and the second rigid section, and
wherein the plurality of coils includes a single layer of coiled metallic wires or multiple layers of coiled metallic wires.

13. The tubular drive shaft according to claim 12,
wherein the second flexible section includes a plurality of coils which are arranged along the tubular body distally to the second rigid section,
wherein the plurality of coils in the second flexible section includes one or more layers of wire coils arranged substantially concentric with the longitudinal axis.

14. The tubular drive shaft according to claim 13,
wherein the plurality of coils in the second flexible section includes two layers of wire coils including a first clockwise wound coil layer and a second counterclockwise wound coil layer.

15. The tubular drive shaft according to claim 13,
wherein the wire coils in the second flexible section include round or circular cross-section wires, square or rectangular cross-section wires, or a combination of round or circular cross-section and square or rectangular cross-section wires.

16. The tubular drive shaft according to claim 1,
wherein the first rigid section, the first flexible section and the second rigid section are made of a single polymeric tube, and
wherein the first flexible section includes a plurality of slots cut through the wall of the tubular body in a section of the single polymeric tube.

17. The tubular drive shaft according to claim 1,
wherein the first rigid section, the first flexible section and the second rigid section are made of a single metallic tube, and
wherein the first flexible section includes a plurality of slots cut through the wall of the tubular body in a section of the single metallic tube.

18. The tubular drive shaft according to claim 1,
wherein the first rigid section, the first flexible section and the second rigid section have a first outer diameter (OD1), and
wherein the second flexible section and the third rigid section have a second outer diameter (OD2), wherein OD2 is smaller than OD1.

19. The tubular drive shaft according to claim 1,
wherein the first flexible section is configured to provide flexible coupling and alignment between the tubular drive shaft and the torque input apparatus, and
wherein a length of the first flexible section is in a range of about 2 millimeters to about 3 centimeters.

20. A tubular drive shaft for a medical device, comprising:
a tubular body having inner and outer surfaces defining a wall that encloses a substantially circular opening that extends along a longitudinal axis from a proximal end to a distal end;
wherein, in order from the proximal end to the distal end, the tubular body includes a first rigid section, a first flexible section having a plurality of slots cut into the wall of the tubular body, a second rigid section, a second flexible section, a third rigid section, a third flexible section, a fourth rigid section,
wherein the first rigid section is configured to couple the tubular drive shaft to a torque input apparatus,
wherein the first flexible section is configured to accommodate alignment of the tubular body with the torque input apparatus, the second flexible section includes a plurality of coils which are arranged along the tubular body distally to the second rigid section between the second rigid section and the third rigid section, the third flexible section includes a plurality of coils which are arranged along the tubular body distally to the third rigid section,
wherein the first flexible section is less flexible than both the second flexible section and the third flexible section,
wherein the fourth rigid section is a metallic tube attached to one or more coils at the distal end of the third flexible section, and
wherein the distal end of the metallic tube includes a transparent window and an atraumatic cap made of radiopaque material.
